(19) United States
(12) Patent Application Publication
(10) Pub. No.: US 2004/0082661 A1
(43) Pub. Date: Apr. 29, 2004

(54) Urea derivatives having vanilloid receptor (vr1) antagonist activity

(76) Inventors: Harshad Kantilal Rami, Harlow (GB); Mervyn Thompson, Harlow (GB); Paul Adrian Wyman, Harlow (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/471,393

(22) PCT Filed: Mar. 7, 2002
(86) PCT No.: PCT/GB02/01046

(30) Foreign Application Priority Data
Mar. 9, 2001 (GB) 0105895.7

(51) Int. Cl. A61K 31/17

(52) U.S. Cl. 514/598; 564/51

(57) ABSTRACT

The invention relates to novel compounds having Vanilloid Receptor (VR1) antagonist activity, processes for their preparation, to compositions containing them and to their use in the treatment of various disorders.

This invention relates to novel compounds in particular novel urea derivatives having pharmacological activity, processes for their preparation, to compositions containing them and to their use in the treatment of various disorders.

Vanilloids are a class of natural and synthetic compounds which are characterised by the presence of a vanillyl (3-Hydroxy 4-methoxyphenyl) group or a functionally equivalent group. The vanilloid Receptor (VR1), whose function is modulated by such compounds, has been widely studied and is extensively reviewed by Szallasi and Blumberg (The American Society for Pharmacology and Experimental Therapeutics, 1999, Vol. 51, No. 2.).

A wide variety of Vanilloid compounds of different structures are known in the art, for example those disclosed in EP 347000, EP 401903, GB 2226313 and WO 92/09285. Particularly notable examples of vanilloid compounds or vanilloid receptor modulators are capsaicin, namely trans 8-methyl-N-vanillyl-6-nonenamide, isolated from the pepper plant, capsazepine (Tetrahedron, Vol. 53, No. 13, pp. 4791-4814, 1997) and olvanil—N-(3-methoxy-4-hydroxy-benzyl)oleamide (J. Med. Chem. 1993, 36, 2595-2604). Recently, certain vanilloid receptor antagonists have been disclosed in WO02/08221.

A structurally novel class of compounds has now been found which also possess Vanilloid receptor (VR1) antagonist activity. The present invention therefore provides, in a first aspect, a compound of formula (I) or a pharmaceutically acceptable salt thereof:

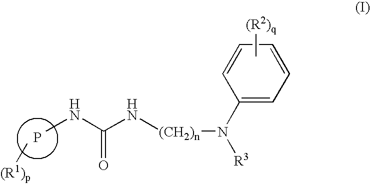

wherein:

P is phenyl or naphthyl;

R¹ is halogen, alkyl, CF₃, hydroxy, alkyloxy, CN, OCF₃, alkylthio, alkylsulfinyl, alkylsulfonyl, nitro, amino, mono- or dialkylamino or C(O)alkyl;

p is 0, 1, 2 or 3;

n is 2, 3, 4, 5 or 6;

R² is halogen, alkyl, CF₃, alkoxy, CN, nitro, aryl, OCF₃, C(O)alkyl, amino, mono- or dialkylamino;

q is 0, 1, 2 or 3;

R³ is hydrogen, alkyl or arylalkyl.

Suitable alkyl groups are C₁₋₆alkyl groups.

When used herein “alkyl” whether used alone or as part of another group refers to straight chain or branched chain alkyl groups.

The term ‘halogen’ is used herein to describe, unless otherwise stated, a group selected from fluorine, chlorine, bromine or iodine.

The term ‘aryl’ is used herein to describe, unless otherwise stated, a group such as phenyl or naphthyl. Such aryl groups may be optionally substituted by one or more C₁₋₆alkyl or halogen.

The term ‘naphthyl’ is used herein to denote, unless otherwise stated, both naphthalen-1-yl and naphthalen-2-yl groups.

When P is naphthyl a preferred group is naphthalen-1-yl. Preferably P is phenyl.

When p is one or more, R¹ is preferably halogen, C₁₋₆alkyl (particularly methyl), C₁₋₆alkoxy (particularly methoxy), C₁₋₆alkylthio (particularly thiomethyl), C(O)C₁₋₆alkyl (particularly acetyl), nitro, CF₃, CN or OCF₃.

When p is 2 or 3 the groups R¹ may be the same or different. Preferably p is 1 or 2.

Preferably n is 2 or 3, most preferably 2.

When q is one or more, R² is preferably halogen, C₁₋₆alkyl (particularly methyl), C₁₋₆alkoxy (particularly methoxy), CF₃, CN or aryl (particularly phenyl).

When q is 2 or 3 the groups R² may be the same or different. Preferably q is 1 or 2. Most preferably q is 1 and R² is a methyl group substituted at the 3 position on the phenyl ring.

When R³ is alkyl, a particularly preferred group is ethyl. When R³ is arylalkyl preferred groups include benzyl or 2-phenethyl.

A particularly preferred compound of this invention is N-[2-bromophenyl]-N′-[2-(N″-ethyl-N″-(3-methylphenyl)amino)ethyl]urea or a pharmaceutically acceptable salt thereof. Other preferred compounds of this invention include examples E1, E2, E5, E13, E14, E16, E17, E21, E28, E29 and E30 (as referenced in Table 1 below) or a pharmaceutically acceptable salt thereof.

Suitably, R¹ is halogen.

Suitably, R² is halogen or alkyl (such as methyl).

The compounds of the formula (I) can form acid addition salts with acids, such as conventional pharmaceutically acceptable acids, for example maleic, hydrochloric, hydrobromic, phosphoric, acetic, fumaric, salicylic, citric, lactic, mandelic, tartaric and methanesulphonic.

Compounds of formula (I) may also form solvates such as hydrates, and the invention also extends to these forms. When referred to herein, it is understood that the term ‘compound of formula (I)’ also includes these forms.

Certain compounds of formula (I) are capable of existing in stereoisomeric forms including diastereomers and enantiomers and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The present invention also provides, in a further aspect, a process for the preparation of a compound of formula (I) or a pharmaceutically acceptable salt thereof, which process comprises coupling a compound of formula (II):

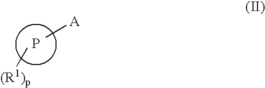

in which R¹, P and p are as defined in formula (I) with a compound of formula (III):

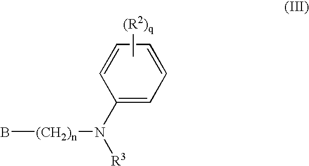

in which R², R³, n and q are as defined in formula (I) and A and B contain the appropriate functional groups which are capable of reacting together to form the urea moiety, and thereafter carrying out one or more of the following optional steps:

(1) removing any protecting group;

(2) converting R¹ into another R¹ or R² into another R² or R³ into another R³; and

(3) forming a pharmaceutically acceptable salt of a compound of formula (I).

Suitable examples of appropriate A and B groups include:

(a) A is —N═C═O and B is NH₂; or

(b) A is NH₂ and B is NH₂;

(c) A is NH₂ and B is N═C═O.

In process (a) or (c), that is when A is —N═C═O and B is NH₂ or vice versa, the reaction is carried out in an inert solvent such as dichloromethane or acetonitrile.

In process (b) the reaction is preferably carried out in the presence of an appropriate urea forming agent, such as carbonyl diimidazole or phosgene, a suitable solvent being an inert organic solvent such as dimethylformamide, tetrahydrofuran, or dichloromethane at ambient or elevated temperature optionally in the presence of a base such as triethylamine or pyridine.

An alternative method of synthesis of the unsymmetrical urea compounds of formula (I) is from a diaryl carbonate, via the corresponding carbamate. Such a methodology is described by Freer et al. (Synthetic Communications, 26(2), 331-349, 1996). It would be appreciated by those skilled in the art that such a methodology could be readily adapted for preparation of the compounds of formula (I).

The above mentioned optional proces steps (1), (2) or (3) are carried out using the appropriate conventional methods, for example those disclosed in standard reference texts such as Comprehensive Organic Transformations, R. C. Larock, Wiley-VCH (Chichester), 1999.

Those skilled in the art will appreciate that it may be necessary to protect certain groups. Suitable protecting groups and methods for their attachment and removal are conventional in the art of organic chemistry, such as those described in Greene T. W. ‘Protective groups in organic synthesis’ New York, Wiley (1981).

Compounds of formulae (II) and (III) are commercially available or may be prepared according to known methods or analogous to known methods.

Pharmaceutically acceptable salts may be prepared conventionally by reaction with the appropriate acid or acid derivative.

Compounds of formula (I) and their pharmaceutically acceptable salts have Vanilloid receptor antagonist (VR1) activity and are believed to be of potential use for the treatment or prophylaxis of certain disorders such as pain, chronic pain, neuropathic pain, postoperative pain, rheumatoid arthritic pain, osteoarthritic pain, back pain, visceral pain, cancer pain, algesia, neuralgia, migraine, neuropathies, diabetic neuropathy, sciatica, HIV-related neuropathy, post-herpetic neuralgia, fibromyalgia, nerve injury, ischaemia, neurodegeneration, stroke, post stroke pain, multiple sclerosis, respiratory diseases, asthma, cough, COPD, inflammatory disorders, oesophagitis, gastroeosophagal reflux disorder (GERD), irritable bowel syndrome, inflammatory bowel disease, pelvic hypersensitivity, urinary incontinence, cystitis, burns, psoriasis, emesis and pruritus.

Thus the invention also provides a compound of formula (I) or a pharmaceutically acceptable salt thereof, for use as a therapeutic substance, in particular in the treatment or prophylaxis of the above disorders. In particular the invention provides a compound of formula (I) or a pharmaceutically acceptable salt thereof or a solvate thereof for use in the treatment or prophylaxis of chronic and acute pain and urinary incontinence.

The invention further provides a method of treatment or prophylaxis of the above disorders, in mammals including humans, which comprises administering to the sufferer a therapeutically effective amount of a compound of formula (I) or a pharmaceutically acceptable salt thereof.

The present invention also provides a pharmaceutical composition, which comprises a compound of formula (I) or a pharmaceutically acceptable salt thereof and a pharmaceutically acceptable carrier.

A pharmaceutical composition of the invention, which may be prepared by admixture, suitably at ambient temperature and atmospheric pressure, is usually adapted for oral, parenteral, rectal administration or intravesical adminstration to the bladder and, as such, may be in the form of tablets, capsules, oral liquid preparations, powders, granules, lozenges, reconstitutable powders, injectable or infusable solutions, suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. For systemic administration, dosage levels from 0.01 mg to 100 mg per kilogramme of body weight are useful in the treatment of pain. However, as a general guide suitable unit doses may be 0.05 to 1000 mg, more suitably 0.05 to 20, 20 to 250, or 0.1 to 500.0 mg, for example 0.2 to 5 and 0.1 to 250 mg; and such unit doses may be administered more than once a day, for example two or three a day, so that the total daily dosage is in the range of about 0.5 to 1000 mg; and such therapy may extend for a number of weeks or months.

When administered in accordance with the invention, no unacceptable toxicological effects are expected with the compounds of the invention.

The following Examples illustrate the preparation of the compounds of the invention.

### DESCRIPTION 1

N-ethyl-N-(3-Fluorophenyl)ethylenediamine

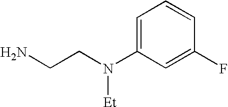

N-Ethyl-3-fluoroaniline (9.2 g, 66 mmol) and 2-bromoethylamine hydrobromide (0.5 eq.) was heated at reflux in toluene (100 ml) for 24 h. After cooling solvent was removed under reduced pressure and the residue suspended in diethyl ether (100 ml), washed with aqueous potassium carbonate (20% solution, 2×100 ml). The ether layer was dried over magnesium sulfate, filtered and solvent removed under reduced pressure. Chromatography on silica gel eluting with dichloromethane and methanol (gradient, maximum 10%) afforded the title compound as an oil (3.9 g), MH⁺ 183 (100%)

### DESCRIPTION 2

N-ethyl-N-(3-Fluoro-4-methylphenyl)ethylenediamine

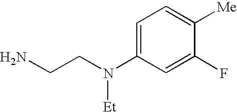

The title compound was prepared from N-ethyl-3-fluoro-4-methylaniline and 2-bromoethylamine hydrobromide according to the procedure outlined in Description 1, MH⁺ 197

### DESCRIPTION 3

N-ethyl-N-(3,4-Difluorophenyl)ethylenediamine

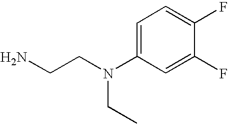

The title compound was prepared from N-ethyl-3,4-difluoroaniline and 1-bromoethylamine hydrobromide according to the procedure outlined in Description 1, MH⁺ 201

### DESCRIPTION 4

N-ethyl-N-(3-Methyl-4-fluorophenyl)ethylenediamine

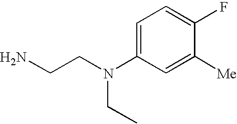

The title compound was prepared from N-ethyl-4-fluoro-3-methylaniline and 2-bromoethylamine hydrobromide according to the procedure outlined in Description 1, MH⁺ 197

### EXAMPLE 1

N-[2-Bromophenyl]-N′-[2-(N″-ethyl-N″-(3-methylphenyl)amino)ethyl]urea

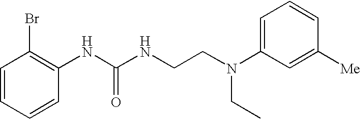

A solution of N-ethyl-N-(3-methylphenyl)ethylenediamine (TCI, Japan) (0.5 g, 2.8 mmol) in DCM (3 ml) was treated with 2-bromophenylisocyanate (Aldrich) (0.57 g, 2.8 mmol) in DCM (2 ml). After stirring the reaction for one hour at room temperature solvent was removed under reduced pressure to afforded the desired product as an off white solid (0.91 g, 86%).

¹H NMR (250 MHz, CDCl₃) δ(ppm): 8.00 (d, 1H), 7.50 (d,1H), 7.26 (m, 1H), 7.10 (m, 1H), 6.92 (m, 1H), 6.55 (m, 4H), 4.95 (br, 1H), 3.47 (m, 4H), 3.37 (q, 2H), 2.30 (s, 3H), 1.14 (t, 3H).

The compounds shown in Table 1 were prepared according to a procedure similar to that of Example E1. All isocyanates used in the synthesis of these Examples are commercially available.

TABLE 1

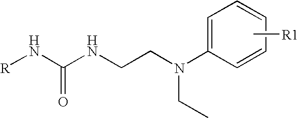

| Example | R | R1 | Observed MH⁺ |
|---|---|---|---|
| E2 | 4-F-Ph | 3-Me | 316 |
| E3 | 3-CN-Ph | 3-Me | 323 |
| E4 | 4-OMe-Ph | 3-Me | 328 |
| E5 | 2-Cl-Ph | 3-Me | 333 |
| E6 | 3,4-diF-Ph | 3-Me | 334 |
| E7 | 3-Ac-Ph | 3-Me | 340 |
| E8 | 3-NO₂-Ph | 3-Me | 341 |
| E9 | 4-SMe-Ph | 3-Me | 342 |
| E10 | 2-Me-3Cl-Ph | 3-Me | 347 |
| E11 | 3-Cl-4-F-Ph | 3-Me | 351 |
| E12 | 3-Cl-4-Me-Ph | 3-Me | 347 |
| E13 | 2-OMe-5-Cl-Ph | 3-Me | 362 |
| E14 | 2-OMe-3-Cl-Ph | 3-Me | 362 |
| E15 | 3-CF₃-Ph | 3-Me | 366 |
| E16 | 2,3-diCl-Ph | 3-Me | 367 |
| E17 | 2,5-diCl-Ph | 3-Me | 367 |
| E18 | 2-OCF₃-Ph | 3-Me | 382 |
| E19 | 2-I-Ph | 3-Me | 424 |
| E20 | 1-Naphthyl | 3-Me | 348 |
| E21 | 2-Br-Ph | 3-F | 380 |
| E22 | 4-F-Ph | 3-F | 320 |
| E23 | 2-Cl-Ph | 3-F | 336 |
| E24 | 2-Me-3-Cl-Ph | 3-F | 350 |
| E25 | 1-Naphthyl | 3-F | 352 |
| E26 | 2,3-diCl-Ph | 3-F | 371 |
| E27 | 2,5-diCl-Ph | 3-F | 371 |
| E28 | 2-BrPh | 3-F-4-Me | 395 |
| E29 | 2-BrPh | 3,4-diF | 399 |
| E30 | 2-BrPh | 3-Me-4-F | 395 |

Pharmacological Data

As referenced above, the compounds of the invention are vanifloid receptor (VR1) antagonists and hence have useful pharmaceutical properties. Vanilloid receptor (VR1) antagonist activity can be confirmed and demonstrated for any particular compound by use of conventional methods, for example those disclosed in standard reference texts such as D. Le Bars, M. Gozarin and S. W. Cadden, Pharmacological Reviews, 2001, 53(4), 597-652] or such other texts mentioned herein. The screen used for the compounds of this invention was derived froma FLIPR based calcium assay, similar to that described by Smart et al. (British Journal of Pharmacology, 2000, 129, 227-230).

Transfected astrocytoma 1321N1 cells, stably expressing human VR1, were seeded into FLIPR plates at 25,000 cells/well (96-well plate) and cultured overnight. The cells were subsequently loaded in medium containing 4 μM Fluo-3 AM Molecular Probes) for 2 hours, at room temperature, in the dark. The plates were then washed 4 times with Tyrode containing 1.5 mM calcium, without probenecid.

The cells were pre-incubated with compound or buffer control at room temperature for 30 minutes. Capsaicin (Sigma) was then added to the cells. Compounds having antagonist activity against the human VR1 were identified by detecting differences in fluorescence when measured after capsaicin addition, compared with no compound buffer controls. Thus, for example, in the buffer control capsaicin addition results in an increase in intracellular calcium resulting in fluorescence. A compound having antagonist activity blocks the capsaicin binding to the receptor, there is no signalling and therefore no increase in intracellular calcium levels and consequently lower fluorescence. pKB values are generated from the IC₅₀ values using the Cheng-Prusoff equation.

All compounds tested by the above methodology had pKb>6, preferred compounds having a pKb>7.0.

1. A compound of formula (I) or a pharmaceutically acceptable salt thereof: 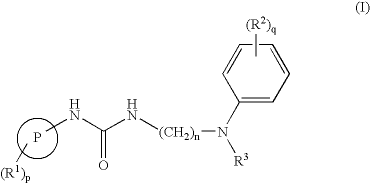
wherein:
P is phenyl or naphthyl;
R¹ is halogen, alkyl, CF₃, hydroxy, alkyloxy, CN, OCF₃, alkylthio, alkylsulfinyl, alkylsulfonyl, nitro, amino, mono- or dialkylamino or C(O)alkyl;
p is 0, 1, 2 or 3;
n is 2, 3, 4, 5 or 6;
R² is halogen, alkyl, CF₃, alkoxy, CN, nitro, aryl, OCF₃, C(O)alkyl, amino, mono- or dialkylamino;
q is 0, 1, 2 or 3;
R³ is hydrogen, alkyl or arylalkyl.

2. A compound according to claim 1 in which P is phenyl.

3. A compound according to claim 1 or claim 2 in which n is 2.

4. A compound according to any of the preceding claims in which R³ is ethyl.

5. A compound according to claim 1 which is: N-[2-bromophenyl]-N′-[2-(N″-ethyl-N″-(3-methylphenyl)amino)ethyl]urea or a pharmaceutically acceptable salt thereof

6. A process for the preparation of a compound of formula (I) or a pharmaceutically acceptable salt thereof, which process comprises coupling a compound of formula (II): 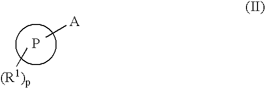
in which R¹, P and p are as defined in formula (I) with a compound of formula (III): 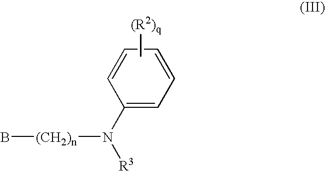
in which R², R³, n and q are as defined in formula (I) and A and B contain the appropriate functional groups which are capable of reacting together to form the urea moiety; and thereafter carrying out one or more of the following optional steps:
(1) removing any protecting group;
(2) converting R¹ into another R¹ or R² into another R² or R³ into another R³; and
(3) forming a pharmaceutically acceptable salt of a compound of formula (I).

7. A compound according to any one of claims 1 to 5 for use in therapy.

8. A compound according to any one of claims 1 to 5 for use in the treatment or prophylaxis of a disorder selected from the list consisting of: pain, chronic pain, neuropathic pain, postoperative pain, rheumatoid arthritic pain, osteoarhritic pain, back pain, visceral pain, cancer pain, algesia, neuralgia, migraine, neuropathies, diabetic neuropathy, sciatica, HIV-related neuropathy, post-herpetic neuralgia, fibromyalgia, nerve injury, ischaemia, neurodegeneration, stroke, post stroke pain, multiple sclerosis, respiratory diseases, asthma, cough, COPD, inflammatory disorders, oesophagitis, gastroeosophagal reflux disorder (GERD), irritable bowel syndrome, inflammatory bowel disease, pelvic hypersensitivity, urinary incontinence, cystitis, bums, psoriasis, emesis and pruritus.

9. A method for the treatment or prophylaxis a disorder selected from the list consisting of: pain, chronic pain, neuropathic pain, postoperative pain, rheumatoid artritic pain, osteoarthritic pain, back pain, visceral pain, cancer pain, algesia, neuralgia, migraine, neuropathies, diabetic neuropathy, sciatica, HIV-related neuropathy, post-herpetic neuralgia, fibromyalgia, nerve injury, ischaemia, neurodegeneration, stroke, post stroke pain, multiple sclerosis, respiratory diseases, asthma, cough, COPD, inflammatory disorders, oesophagitis, gastroeosophagal reflux disorder (GERD), irritable bowel syndrome, inflammatory bowel disease, pelvic hypersensitivity, urinary incontinence, cystitis, burns, psoriasis, emesis and pruritus, in mammals including humans, which comprises administering to the sufferer a therapeutically effective amount of a compound of formula (I) according to claim 1, or a pharmaceutically acceptable salt thereof.

10. A pharmaceutical composition which comprises a compound according to any one of claims 1 to 5 and a pharmaceutically acceptable carrier or excipient.
